(19) United States
(12) Plant Patent Application Publication
(10) Pub. No.: US 2014/0041088 P1
(43) Pub. Date: Feb. 6, 2014

(54) Hemerocallis plant named 'Connie Marks'

(50) ;

(71) Applicant: Randy Marks, Culpeper, VA (US)

(72) Inventor: Randy Marks, Culpeper, VA (US)

(21) Appl. No.: 13/507,872

(22) Filed: Aug. 3, 2012

(51) Int. Cl. A01H 5/00

(52) U.S. Cl. PLT/312

(57) ABSTRACT

A new and distinct cultivar of Hemerocallis named “Connie Marks” characterized by double, light yellow, fragrant flowers that are nocturnal, fully double, lightly creped and ruffled, 13.4 cm in diameter, and of considerable substance that blooms for 24 hours. ‘Connie Marks’ has a propensity to form a plurality of scapes per fan and two per bloom period fan, is heat and cold hardy to a wide range of Hardiness Zones—U.S.D.A. zones No. 3 to No. 7, has a delicate perfumed fragrance, and an extended bloom period from early June until the end of July which is longer than a thirty day or less average for more than 99% of cultivates currently in the trade, ‘Connie Marks’ cleanly sheds spent flowers to allow new buds to fully develop, readily propagates by division and exhibits a moderate rate of multiplication of three or more fans per year, and is resistant to daylily rust (puccinia hemerocallidis.)

### BOTANICAL CLASSIFICATION

Hemerocallis mutation

### CULTIVAR DESIGNATION

‘Connie Marks’

### BACKGROUND OF THE DISCLOSURE

A new and distinct Hemerocallis cultivar ‘Connie Marks’, herein after also referred to as “the plant”, is a new and distinct plant variety that has been asexually reproduced true to type by division in successive generations. It was discovered in a cultivated area when it first bloomed during the summer of 2001 at a commercial nursery/garden center located in Culpeper, Va. The plant ‘Connie Marks’ was an asexual offspring of an American Hemerocallis Society registered daylily named ‘Hyperion’ which was registered in 1925. Asexual offspring refers to the fact that ‘Hyperion’ was the only other daylily growing in the nursery garden vicinity where ‘Connie Marks’ was discovered. The large ‘Hyperion’ clump had a smaller 6 fan, double yellow flowered ‘Connie Marks’ clump growing on its perimeter. ‘Hyperion's’ flowers are single. The plant has been successfully propagated by division methods at the same nursery to produce identical plants that maintain the unique characteristics of the original. Since 2001 the original 6 fan ‘Connie Marks’ has been repeatly divided to produce numerous plants that are phenotypically identical.

Hemerocallis hybrida ‘Hyperion’ is the vegetative parent of ‘Connie Marks’, which is a mutation. There were no daylilies, except ‘Hyperion,’ growing in the larger nursery garden and due to physical structures of the nursery and surrounding arborvitae hedge, with no daylilies outside of the hedge, normal prevailing winds would not indicate pollen was windborne from a distant site.

### SUMMARY OF THE INVENTION

The following traits have been repeatedly observed and represent the characteristics of ‘Connie Marks.’ These attributes, in combination, distinguish ‘Connie Marks’ as a new and distinct cultivar:

(1) ‘Connie Marks’ is a nocturnal, fully double, lightly creped and ruffled, 13.4 cm in diameter, light yellow daylily of considerable substance that blooms for 24 hours;

(2) ‘Connie Marks’ has a propensity to form a plurality of scapes per fan, two scapes per fan per bloom period;

(3) ‘Connie Marks’ is heat and cold hardy to a wide range of Hardiness Zones—U.S.D.A. zones No. 3-No. 7;

(4) ‘Connie Marks’ has a delicate perfumed fragrance;

(5) ‘Connie Marks’ has an extended bloom period from early June until the end of July which is longer than a thirty day average bloom period for more than 99% of cultivars currently in the trade which have bloom periods of less than thirty days;

(6) ‘Connie Marks’ cleanly sheds spent flowers to allow new buds to fully develop;

(7) The plant readily propagates by division and exhibits moderate rate of multiplication of three or more fans per fan per year; and

(8) ‘Connie Marks’ is resistant to daylily rust—puccinia hemerocallidis.

Plants of the cultivar ‘Connie Marks’ can be compared to the parent cultivar Hemerocallis hybrida ‘Hyperion’ (unpatented). Plants of the instant cultivar ‘Connie Marks’ and plants of the cultivar ‘Hyperion’ differ in the following characteristics:

1. Plants of ‘Connie Marks’ have double flowers whereas plants of ‘Hyperion’ have single flowers.

2. Plants of ‘Connie Marks’ have smaller flowers than plants of ‘Hyperion’.

3. Plants of ‘Connie Marks’ have petaloids with anthers, whereas plants of ‘Hyperion’ have the more typical anthers attached to filaments.

4. Plants of ‘Connie Marks’ exhibit more creping on the inner tepals than plants of ‘Hyperion’.

### BRIEF DESCRIPTION OF THE PHOTOGRAPHS

The accompanying photographs of the new variety ‘Connie Marks’ demonstrate the overall appearance of the plant, including several unique traits. The colors shown are as accurate as reasonably possible with color reproductions of this type. Ambient light spectrum, source and direction may cause the appearance of minor variation in colors but in general the dominant color is a light lemon yellow. Color values cited in the detailed botanical description more accurately describe the actual colors of ‘Connie Marks’.

FIG. 1 shows a close-up frontal view of a typical plant of ‘Connie Marks’ with a clump of several flowers, buds, scapes, stems, etc. visible;

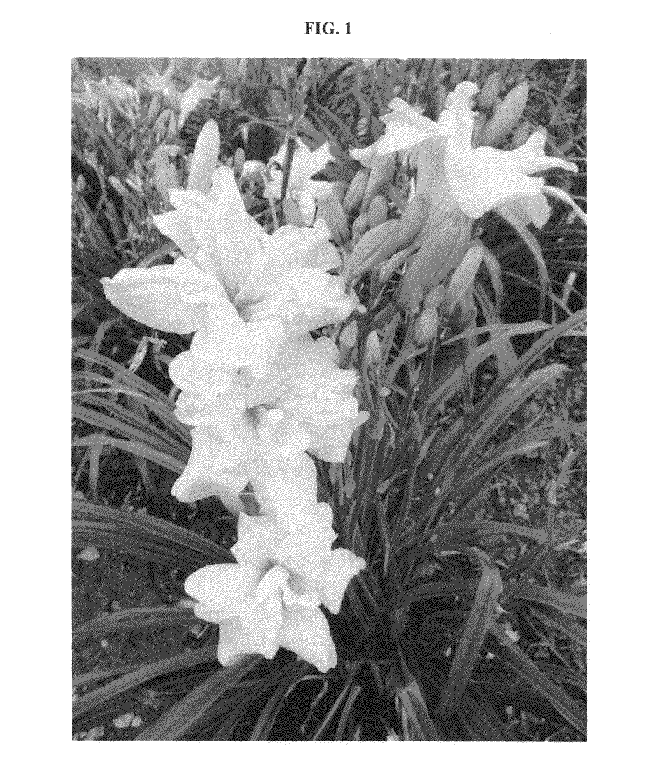

FIG. 2 shows a frontal view of a typical blossom of ‘Connie Marks’; and

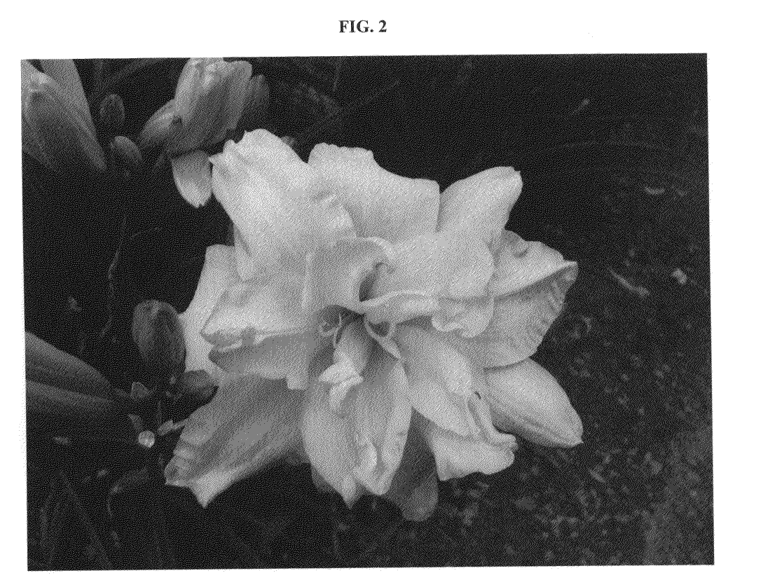

FIG. 3 shows some of the unique sexual structures of ‘Connie Marks’, including petaloids with anthers.

### DETAILED BOTANICAL DESCRIPTION

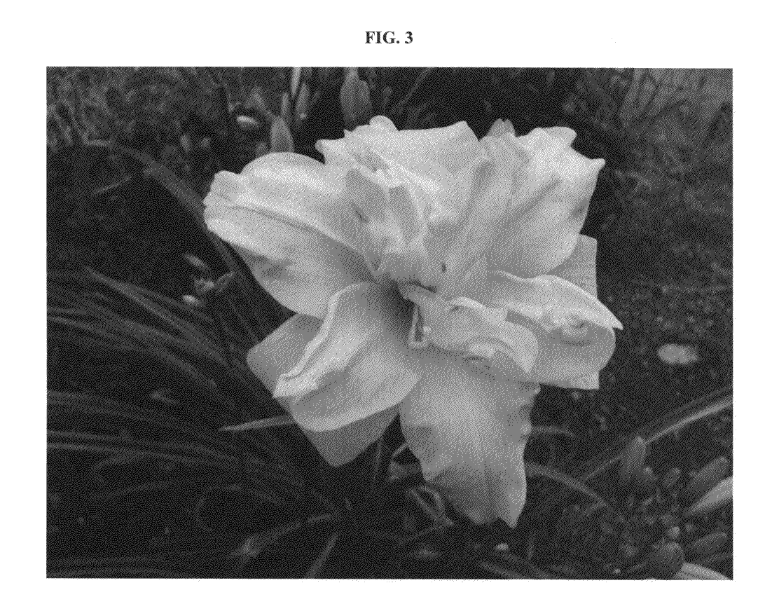

The following is a detailed description of a three year old plant of the new cultivar as grown outdoors in a three gallon container in Culpeper, Va. The color determination is in accordance with the 2007 R.H.S. Colour Chart of The Royal Horticultural Society, London, England, except where general color terms of ordinary dictionary significance are used.
General characteristics:
   Bloom period.—Early June until end of July.
   Plant habit.—Herbaceous perennial, grass-like foliage.
   Height and spread.—Hardy, deciduous, winter dormant, herbaceous rhizomatous perennial with basal linear leaves arching in upper ⅔s, natural foliage spread is generally 66 cm in diameter and generally 79 cm tall with an occasional leaf up to 90 cm tall and symmetrical.
   Hardiness.—U.S.D.A. zones No. 3 to 7.
   Disease and pest.—Has been observed to be resistant to daylily rust — Puccinia hemerocallidis.
   Root description.—Fibrous roots.
   Propagation.—Division; and
   Growth rate.—Moderate with three to four fans per fan season.
Foliage description:
   Leaf shape.—Linear.
   Leaf division.—Simple.
   Leaf base.—Sheathed to crown.
   Leaf apex.—Narrowly acute.
   Leaf venation.—Longitudinal, parallel, same color as leaves.
   Leaf margin.—Entire.
   Leaf attachment.—Sheathed, not petioled.
   Leaf arrangement.—Fan shaped.
   Leaf surface.—Glabrous (without hairs).
   Leaf color.—Mature leaves (upper and lower surface) R.H.S. Green Group upper leaf 137B and lower leaf 137C.
   Leaf size.—Up to 90 cm in length, 0.5 to 2 cm wide; and
   Leaf quantity.—approximately 150 leaves to 3 gallon pot.
Flower scape description:
   Scape shape.—In approximately equal proportions scapes are ovoid, square, and round and ovoid at the base and all scapes are hollow.
   Scape number.—Two per fan at one time.
   Scape aspect.—Straight, held upright.
   Scape size.—An average of 63.5 cm in length and 8 mm in width, branches average 3 cm in length and 4 mm in width.
   Scape color.—R.H.S 138A, Green.
   Scape surface.—Glabrous.
   Scape branching.—Average three branches per scape, internode length 2 cm; and
   Scape leaves.—One per branch point plus two additional not at branch points, shortest at 1.2 cm long up to longest at 6.8 cm, not at branch, but on shaft, all leaves are glabrous.
Flower description:
   Inflorescence type.—Scape bearing an average of one or more double flowers simultaneously.
   Inflorescence size.—An average of 6 cm deep and 13.4 cm in diameter.
   Flower duration.—About twenty four hours, self cleaning.
   Flower number.—Average of twelve per scape.
   Flower aspect.—Upward to outward.
   Flower color.—Apex: R.H.S. 144C, Yellow-Green; longitudinally RHS 150B, Yellow-Green; longitudinal edges RHS 154A Yellow-Green.
   Flower bud shape.—Oblanceolate, broadest width above the middle.
   Flower attachment.—Petiolate.
   Petal color.—R.H.S. 138A, Green; both sides, base has a light green throat, RHS 154B, Yellow-Green.
   Petal number.—The average number of petals and petaloids is thirteen point five and can be as much as sixteen.
   Petal shape.—Broadly lanceolate with petaloid variety.
   Petaloid color.—R.H.S. 7A, Yellow, with slight base, throat of RHS 154B, Yellow-Green.
   Petal surface.—Glabrous, waxy, mid-vein raised in upper surface, creped texture.
   Petal margins.—Entire, and lightly ruffled, slightly irregular.
   Petal apex.—Slightly irregular and rounded, obtuse.
   Petal size.—Average width 4.3 cm and 10 cm long.
   Petal aspect.—Slightly recurved.
   Sepal number.—50% three and 50% four.
   Sepal shape.—Elliptical to narrowly lanceolate, moderately recurved.
   Sepal margin.—Entire, very slightly ruffled.
   Sepal size.—Width in the middle 2.3 cm and 10.3 cm long.
   Sepal aspect.—Moderately recurved.
   Sepal surface.—Glabrous, smooth surface.
   Sepal apex.—Acute.
   Sepal base.—Fused.
   Sepal color.—Both surfaces R.H.S. 7A, Yellow base with RHS 154B, Yellow-Green to form slightly green throat.
   Pedicel size.—Average 1.0 cm in length and 4 mm in width.
   Pedicel shape.—Ovoid, three sided.
   Pedicel color.—R.H.S. 144A, Yellow-Green; and
   Pedicel strength/aspect.—Moderately strong, held at about 20° relative to peduncle.
Reproductive organs: (1) Gynoecium (female) Pistil varies with some pistils corkscrewing around inside flower, some very short (3 cm) and an average length of 7.8 cm and 1 mm in width with color RHS12C, Yellow, some are longer with multiple branches near the apex, less than 40% have an average length of about 9 cm and a width on average 1 mm, approximately 90% of ovaries are 5 mm in length and an average width of 4 mm and color RHS 138B, Green; ovules base color RHS 11B, Yellow, apex (Stigma) RHS 12C, Yellow (note particularly FIG. 3). No fruit or seeds were observed.
(2) Androcoecium (male)
   Stamen (anther and filament).—the number of filament anthers and petaloid anthers varies with the greatest number observed being six petaloid anthers and three filament anthers, petaloid color RHS 7A, Yellow (note again FIG. 3).
   Pollen.—color RHS 16A, Yellow-Orange, moderate in quantity for anthers.
   Stamens.—Average filament length 6.8 cm, width 1 mm, filament color RHS 11A, Yellow, anthers measure on average a total of 6.17 mm in length for anthers with color RHS 17A, Yellow-Orange.
Fragrance: Light perfume like fragrance similar to cultivar ‘Hyperion’. Habitat in Culpeper, Va. experiences a normal summer; daytime high temperature ranges between 24°-34° C., with a nighttime low temperature range of 9° to 20° C.

What is claimed is:

1. A new and distinct cultivar of Hemerocallis plant named ‘Connie Marks’ as illustrated and described herein.
